(19) United States
(12) Patent Application Publication
(10) Pub. No.: US 2023/0050245 A1
(43) Pub. Date: Feb. 16, 2023

(54) METHODS AND SYSTEMS FOR DETERMINING AND DISPLAYING PATIENT READMISSION RISK

(71) Applicant: KONINKLIJKE PHILIPS N.V., Eindhoven (NL)

(72) Inventor: Eran Simhon, Cambridge (NL)

(21) Appl. No.: 17/879,806

(22) Filed: Aug. 3, 2022

Related U.S. Application Data
(60) Provisional application No. 63/229,124, filed on Aug. 4, 2021.

(51) Int. Cl. G16H 50/30; G16H 10/60; G06N 5/02

(52) U.S. Cl. CPC G16H 50/30; G16H 10/60; G06N 5/022

(57) ABSTRACT

A method for generating and presenting a patient readmission risk using a readmission risk analysis system, comprising: (i) receiving information about the patient, wherein the information comprises a plurality of readmission prediction features; (ii) extracting the plurality of readmission prediction features from the received information; (iii) analyzing the readmission prediction features to determine whether each of a predetermined list of readmission prediction features are present; (iv) replacing one or more identified missing readmission prediction features with a null value to generate a complete set of readmission prediction features for the patient; (v) analyzing the complete set of readmission prediction features for the patient to generate a readmission risk score; (vi) determining, using a populated lookup table of the readmission risk analysis system, an AUC score; and (vii) displaying the generated readmission risk score and the determined AUC score.

### FIELD OF THE DISCLOSURE

The present disclosure is directed generally to methods and systems for predicting patient readmission risk using a readmission risk analysis system.

### BACKGROUND

Patient readmission occurs when a patient is readmitted to a hospital or other care facility within a certain period of time (e.g., days or weeks) after having been discharged, usually for treatment of the same or related condition. Readmission rates can be especially high for certain conditions. However, readmission can be unfavorable for several reasons. For example, high readmission rates may reflect upon the quality of the care provided by the hospital or care facility during the previous admission. Additionally, readmission can have significant financial consequences for the care facility. Indeed, readmission is becoming an increasingly important problem in the United States, due in large part to financial incentives and/or penalties from payers discouraging preventable readmissions.

Although high readmission rates can be unfavorable, it is often difficult to identify patients subject to readmission, and/or to identify the in-facility treatment or post-discharge care that best prevents readmission. It is therefore useful to predict readmission probability for a given patient, and to identify interventions both within and outside the hospital that could potentially reduce a patient's risk or need for readmission.

Existing systems or methods for identifying readmission probabilities or risk have significant limitations. For example, these systems typically do not support missing data and even when they do support missing data, there is no estimation of accuracy when there is one or more missing features. Existing systems also fail to utilize International Classification of Diseases (ICD) codes, the globally used standard of diagnostic codes for classifying diseases, when identifying readmission probabilities or risk. Additionally, existing systems typically require extensive information about a patient in order to determine readmission probability or risk.

### SUMMARY OF THE DISCLOSURE

Accordingly, there is a continued need for methods and systems that predict patient readmission risk and that support missing data, that can utilize ICD codes, and that requires limited information about the patient.

Various embodiments and implementations herein are directed to a method and system configured to generate and present a patient readmission probability or risk using a readmission risk analysis system. The system receives information about the patient, comprising a plurality of readmission prediction features. The system then extracts the plurality of readmission prediction features from the received information, and analyzes these features to determine whether each of a predetermined list of readmission prediction features are present. The system replaces one or more identified missing readmission prediction features with a null value to generate a complete set of readmission prediction features for the patient. A trained machine learning algorithm of the risk score analysis system analyzes the complete set of readmission prediction features to generate a readmission risk score. The system determines an AUC score for the data using a populated lookup table of the readmission risk analysis system. The pre-populated lookup table comprises an AUC score for a complete set of readmission prediction features when the complete set of readmission prediction features comprises the one or more identified missing readmission prediction features. The system then displays, via a user interface of the readmission risk analysis system, the generated readmission risk score, and the determined AUC score. The display also includes the effect of one or more of the individual readmission prediction features in the complete set of readmission prediction features on the generated readmission risk score, such as the top three features that have the largest impact on the readmission risk score.

Generally, in one aspect, a method for generating and presenting a patient readmission risk using a readmission risk analysis system is provided. The method includes: (i) receiving, at the readmission risk analysis system, information about the patient, wherein the information comprises a plurality of readmission prediction features; (ii) extracting, by a processor of the readmission risk analysis system, the plurality of readmission prediction features from the received information; (iii) analyzing, by the processor, the extracted plurality of readmission prediction features to determine whether each of a predetermined list of readmission prediction features are present; (iv) replacing one or more identified missing readmission prediction features with a null value to generate a complete set of readmission prediction features for the patient; (v) analyzing, using a trained machine learning algorithm of the risk score analysis system, the complete set of readmission prediction features for the patient to generate a readmission risk score; (vi) determining, using a populated lookup table of the readmission risk analysis system, an AUC score, wherein the populated lookup table comprises an AUC score for a complete set of readmission prediction features when the complete set of readmission prediction features comprises the one or more identified missing readmission prediction features; and (vii) displaying, via a user interface of the readmission risk analysis system, the generated readmission risk score and the determined AUC score.

According to an embodiment, the display further comprises an effect of one or more of the individual readmission prediction features in the complete set of readmission prediction features on the generated readmission risk score. According to an embodiment, the display of the effect of one or more of the individual readmission prediction features on the generated readmission risk score comprises a ranked list of: (1) one or more individual readmission prediction features on with a highest effect increasing the readmission risk score for the patient; or (2) one or more readmission prediction features on with a highest effect decreasing the readmission risk score for the patient. According to an embodiment, the highest effect on a higher readmission risk score for the patient or the highest effect on a lower readmission risk score for the patient is determined by comparing the effect to a predetermined threshold.

According to an embodiment, the plurality of readmission prediction features comprises a diagnosis for the patient.

According to an embodiment, the readmission risk score comprises a SHAP value for one or more of the individual readmission prediction features in the complete set of readmission prediction features.

According to an embodiment, displaying further comprises displaying a SHAP value for one or more of the individual readmission prediction features in the complete set of readmission prediction features.

According to an embodiment, the method further comprises training a model of the readmission risk analysis system using a training dataset comprising data about a plurality of patients. For example, the method includes: (i) training a first model of the readmission risk analysis system to generate a first model readmission risk score without a diagnosis information for a patient; (ii) mapping each of a plurality of ICD codes to one or more of a plurality of clinical categories; (iii) computing, from the training dataset, a comorbidity index for each patient in the plurality of patients; (iv) training an intermediate model of the readmission risk analysis system to generate, for each patient, an intermediate clinical category-based readmission risk score using the plurality of clinical categories; and (v) training a second model of the readmission risk analysis system to generate a second model readmission risk score for each of the plurality of patients using, for each patient: (a) a plurality of prediction features extracted from the training data for the respective patient; (b) the generated intermediate clinical category-based readmission risk score for the respective patient; and (c) the computed comorbidity index for the respective patient.

According to an embodiment, the training further includes: estimating an AUC score for all possible combinations of missing values for the plurality of prediction features; and determining a SHAP value for each of the plurality of prediction features.

According to a second aspect, a readmission risk analysis system is provided. The system includes: a trained readmission risk model configured to generate a readmission risk score from a plurality of extracted readmission prediction features about a patient; a processor configured to: (i) receive information about the patient, wherein the information comprises a plurality of readmission prediction features; (ii) extract the plurality of readmission prediction features from the received information; (iii) analyze the extracted plurality of readmission prediction features to determine whether each of a predetermined list of readmission prediction features are present; (iv) replace one or more identified missing readmission prediction features with a null value to generate a complete set of readmission prediction features for the patient; (v) analyze, using the trained readmission risk model, the complete set of readmission prediction features for the patient to generate a readmission risk score; and (vi) determining an AUC score; and a user interface configured to present to a user the generated readmission risk score and AUC score for the patient.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for predicting patient readmission risk, in accordance with an embodiment.

FIG. 2 is a schematic representation of a readmission risk analysis system, in accordance with an embodiment.

FIG. 3 is a flowchart of a method for training a readmission risk analysis system, in accordance with an embodiment.

FIG. 4 is a flowchart of a method for training a readmission risk analysis system, in accordance with an embodiment.

FIG. 5 is a schematic representation of an AUC table of a readmission risk analysis system, in accordance with an embodiment.

FIG. 6 is a flowchart of a method for predicting patient readmission risk, in accordance with an embodiment.

FIG. 7 is a schematic representation of an output of a readmission risk analysis system, in accordance with an embodiment.

FIG. 8 is a schematic representation of an output of a readmission risk analysis system, in accordance with an embodiment.

### DETAILED DESCRIPTION OF EMBODIMENTS

The present disclosure describes various embodiments of a system and method configured to generate and present a patient's readmission risk score. More generally, Applicant has recognized and appreciated that it would be beneficial to provide a method and system to prevent patient readmission. Accordingly, a trained readmission risk analysis system quantifies and communicates patient readmission risk to a clinician, which allows the clinician to make decisions about in-facility and/or post-discharge care. The trained readmission risk analysis system receives information about a patient, the information including a plurality of readmission prediction features. These features are extracted from the received information and analyzed by the system to determine whether each of a predetermined list of readmission prediction features are present. Any identified missing readmission prediction features are replaced with a null value or other indicator in order to generate a complete set of readmission prediction features for the patient. This set of features is analyzed using a trained machine learning algorithm to generate a readmission risk score. A populated lookup table of the readmission risk analysis system is utilized to determine an AUC score. This populated lookup table comprises an AUC score for a complete set of readmission prediction features when the complete set of readmission prediction features comprises the one or more identified missing readmission prediction features. The system then displays the generated readmission risk score and the determined AUC score. The system also displays an effect of one or more of the individual readmission prediction features on the generated readmission risk score.

According to an embodiment, the systems and methods described or otherwise envisioned herein can, in some non-limiting embodiments, be implemented as an improvement to existing commercial products for patient analysis or monitoring, such as an Intellivue Guardian bedside monitor, or a Central Station (both available from Koninklijke Philips NV, the Netherlands), or any suitable patient or care facility system.

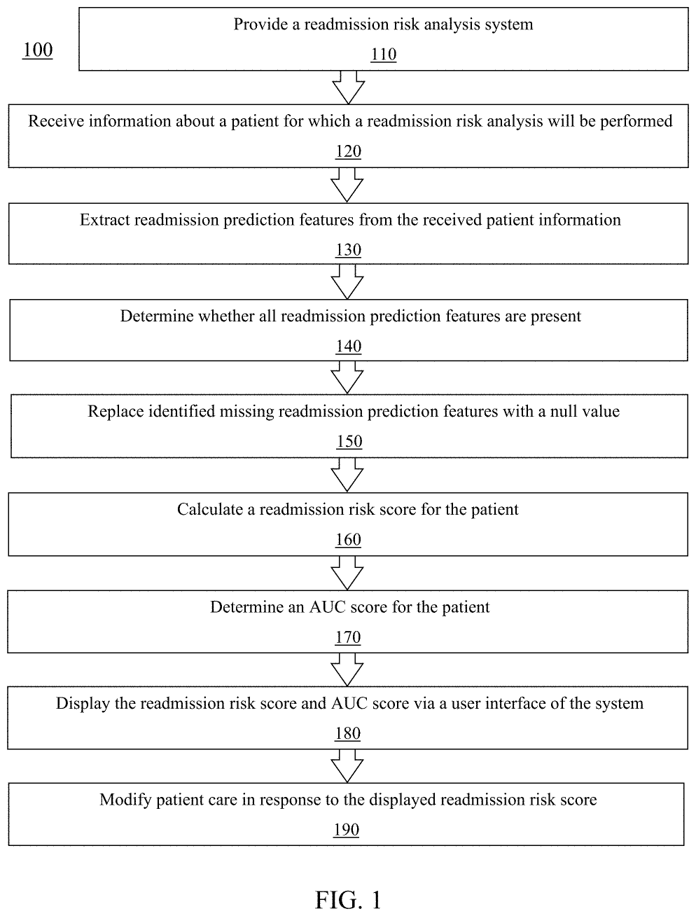

Referring to FIG. 1, in one embodiment is a flowchart of a method 100 for generating and communicating a patient readmission risk using a readmission risk analysis system. The methods described in connection with the figures are provided as examples only, and shall be understood not limit the scope of the disclosure. The readmission risk analysis system can be any of the systems described or otherwise envisioned herein. The readmission risk analysis system can be a single system or multiple different systems.

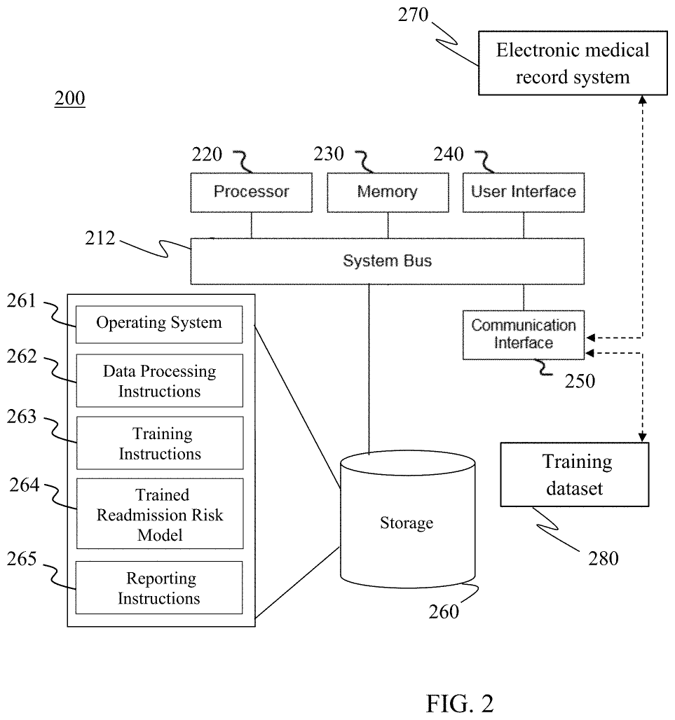

At step 110 of the method, a readmission risk analysis system is provided. Referring to an embodiment of a readmission risk analysis system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, readmission risk analysis system 200 can be any of the systems described or otherwise envisioned herein. Other elements and components of readmission risk analysis system 200 are disclosed and/or envisioned elsewhere herein.

At step 120 of the method, the readmission risk analysis system receives information about a patient for which a readmission risk analysis will be performed. According to an embodiment, the information comprises a plurality of features about the patient. Only some, or all, of the information about the patient may ultimately be utilized by the readmission risk analysis system. The plurality of features may comprise, for example, vital sign information about the patient, including but not limited to physiologic vital signs such as heart rate, blood pressure, respiratory rate, apnea, SpO₂, invasive arterial pressure, noninvasive blood pressure, and more. According to an embodiment, the information may also comprise medical information about the patient, including but not limited to demographics, physiological measurements other than vital data such as physical observations, and/or patient diagnosis, among many other types of medical information. As an example, the medical information can include detailed information on patient demographics such as age, gender, and more; diagnosis or medication condition such as cardiac disease, psychological disorders, chronic obstructive pulmonary disease, and more. Many other types of medical information are possible. Accordingly, the received information can be any information relevant to a patient readmission risk analysis.

According to just one embodiment of the readmission risk analysis method, and provided only as a non-limiting example, the information about the patient comprises one or more of the features listed in Table 1. The patient features ultimately utilized by the readmission risk analysis may comprise just one, some, or all of the features in Table 1, and may further comprise one or more other patient features in addition to the features listed in Table 1.

TABLE 1
Example of Patient Features

| | |
|---|---|
| LOS (int) | The length of stay (e.g., discharge day to admitting day). |
| | According to an embodiment, when predicting the readmission |
| | of current patients, use the current date as the discharge date. |
| num_ED_vists (int) | The number of emergency department (ED) visits in a |
| | predetermined time period, such as the past six months, |
| | although other time periods are possible. According to an |
| | embodiment, this is the number of ED visits that didn't lead to |
| | admission. For example, when implementing on admission- |
| | discharge-transfer (ADT) data, the system will count all |
| | encounters where “Admit_type==Emergency” AND |
| | “Encounter_type!=Inpatient” AND “admission_date − current |
| | date < 183 days,” although other time periods, combinations, |
| | and parameters are possible. |
| acute_admission (int) | According to an embodiment the system sets this value to “1” if |
| | the current admission is acute, otherwise the system sets the |
| | value to “0.” For example, when implementing on admission- |
| | discharge-transfer (ADT) data, the system will set this value to |
| | “1” if “Admit_type != Elective.” |
| age (int) | According to an embodiment, this value is the age of the patient |
| | in years. |
| gender (int) | According to an embodiment, the system sets this value to “1” |
| | if the patient is male and sets the value to “0” if the patient is |
| | female, or vice versa. The system may also comprise other |
| | values or integers for other genders. |
| num_not_elective_admissions | According to an embodiment, this value is the number of |
| (int) | elective admissions in a predetermined prior period, such as a |
| | year (although other time periods are possible). For example, |
| | when implementing on admission-discharge-transfer (ADT) |
| | data, the system will count all encounters where “Admit_type |
| | != Elective” AND “Encounter_type == Inpatient” AND |
| | “admission_date − current date < 365 days,” although other |
| | time periods, combinations, and parameters are possible. |
| num_elective_admissions (int) | According to an embodiment, this value is the number of non- |
| | elective admissions in a predetermined prior period, such as a |
| | year (although other time periods are possible). For example, |
| | when implementing on admission-discharge-transfer (ADT) |
| | data, the system will count all encounters where “Admit_type == |
| | Elective” AND “Encounter_type == Inpatient” AND |
| | “admission_date − current date < 365 days,” although other |
| | time periods, combinations, and parameters are possible. |
| List of ICD codes | According to an embodiment, this value is a list of one or more |
| | ICD codes associated with the current patient for the current |
| | encounter. For example, the value may be a list of all ICD |
| | codes associated with the current patient for the current |
| | encounter. |

The readmission risk analysis system can receive patient information from a variety of different sources, including any source that comprises one or more patient features. According to an embodiment, the readmission risk analysis system is in communication with an electronic medical records database from which the patient information and one or more of the plurality of features may be obtained or received. The electronic medical records database may be a local or remote database and is in communication the readmission risk analysis system 200. According to an embodiment, the readmission risk analysis system comprises an electronic medical record database or system 270 which is optionally in direct and/or indirect communication with system 200. According to another embodiment, the readmission risk analysis system may obtain or receive the plurality of features from equipment or a healthcare professional obtaining that information directly from the patient.

According to an embodiment, the readmission risk analysis system may query an electronic medical record database or system, comprising fast healthcare interoperability resources (FHIR) for example, to obtain the patient information.

At step 130 of the method, the readmission risk analysis system extracts a plurality of readmission prediction features from the patient information received in step 120 of the method. The plurality of readmission prediction features may comprise a predetermined list of patient features, such as the non-limiting example provided in Table 1 although other lists are possible. The system is therefore trained to identify and extract the predetermined list of patient features from the received patient information, using any of a wide variety of algorithms, methods, or systems for identifying and extracting patient data. The plurality of identified and extracted patient features may be utilized immediately or may be stored in local or remote storage for use in further steps of the method.

At step 140 of the method, the readmission risk analysis system analyzes the extracted plurality of readmission prediction features to determine whether each of a predetermined list of readmission prediction features are present in the set of features for the patient. For example, the predetermined list of readmission prediction features may comprise a list of two or more patient features that are utilized by the system to determine a patient's readmission risk. According to an embodiment, the predetermined list of readmission prediction features comprises one, some, or all of the features in Table 1, and may further comprise one or more other patient features in addition to the features listed in Table 1. According to just one non-limiting example, the predetermined list of readmission prediction features comprises the eight patient features listed in Table 1.

According to an embodiment, the readmission risk analysis system analyzes the extracted plurality of readmission prediction features to determine whether each of a predetermined list of readmission prediction features are present in the set of features for the patient, by comparing the extracted plurality of readmission prediction features to a predetermined list of readmission prediction features, where the predetermined list of readmission prediction features comprises those features that will be utilized by the system to perform a patient readmission risk analysis. For example, the predetermined list of readmission prediction features may comprise a list of X number of features that are utilized by the system to perform a patient readmission risk analysis, where X is a variable number depending on the settings, parameters, or training of the readmission risk analysis system.

The analysis or comparison can be made using any of a wide variety of algorithms, methods, or systems for comparing data. The output of the analysis or comparison may be an identification of those readmission prediction features that are present for the patient, those readmission prediction features that are missing for the patient, or both. The output may be utilized immediately or may be stored in local or remote storage for use in further steps of the method.

At step 150 of the method, the readmission risk analysis system has identified in step 140 one or more patient features that are missing from the predetermined list of readmission prediction features utilized to perform a readmission risk analysis, or has identified one or more patient features that are missing usable data. There may be no data, or the data may be unusable for one or more reasons. The readmission risk analysis system then replaces the identified one or more missing patient features with an identifier to generate a complete set of readmission prediction features for the patient. The identifier may be a null value or any other indicator of missing or unusable data. For example, the system may be programmed to recognize the identifier as an indicator of missing or unusable data.

According to an embodiment, the generated complete set of readmission prediction features for the patient comprises a set of predetermined readmission prediction features for the patient which is utilized for the readmission risk analysis. The generated complete set of readmission prediction features for the patient may be utilized immediately or may be stored in local or remote storage for use in further steps of the method.

At step 160 of the method, the readmission risk analysis system generates a readmission risk score for the patient using the generated complete set of readmission prediction features for the patient. According to an embodiment, the readmission risk analysis system utilizes a trained machine learning algorithm or model to generate the readmission risk score for the patient using the generated complete set of readmission prediction features. The readmission risk analysis system may comprise the trained machine learning algorithm or model, or may be in communication with the trained machine learning algorithm or model.

According to an embodiment, the trained machine learning algorithm used by the readmission risk analysis system depends upon the readmission prediction features found within the set of readmission prediction features for the patient. For example, the system may utilize a first algorithm if the set of readmission prediction features comprises a predetermined one or more of the prediction features, and may utilize a second algorithm if the set of readmission prediction features is missing a predetermined one or more of the prediction features. According to one non-limiting example, the readmission risk analysis system utilizes an ICD algorithm if the prediction features comprise one or more ICD codes for the patient, but utilizes a non-ICD algorithm if the prediction features comprise no ICD codes for the patient or comprise unusable ICD codes for the patient. The parameters and training of the machine learning algorithms are described in greater detail herein.

According to an embodiment, generating a readmission risk score for the patient comprises generating, calculating, estimating, or otherwise determining an effect on the generated readmission risk score of one or more of the individual readmission prediction features. For example, the readmission risk analysis system may determine a SHAP (SHapley Additive exPlanation) value for each of the individual readmission prediction features. The SHAP value may be determined using any method for determining SHAP values, or otherwise generating, calculating, estimating, or otherwise determining the effect. The SHAP values may be utilized immediately or may be stored in local or remote storage for use in further steps of the method.

At step 170 of the method, the readmission risk analysis system determines or estimates an area under the curve (AUC) score for the patient using the prediction features, based on the one or more identified missing features from the set of readmission prediction features for the patient. According to an embodiment, the system utilizes a pre-populated lookup table of the readmission risk analysis system to retrieve information and generate an AUC score. The pre-populated lookup table comprises an AUC score for a plurality of sets of readmission prediction features, where each set of the plurality of sets comprises one or more missing readmission prediction features. According to an embodiment, determining or estimating an AUC score when there are two or more missing values or features comprises determining or estimating an AUC score for all combinations of the missing values or features. Generation of the pre-populated lookup table of the readmission risk analysis system is described herein. The determined or estimated AUC score may be utilized immediately or may be stored in local or remote storage for use in further steps of the method.

At step 180 of the method, the readmission risk analysis system displays or otherwise provides the generated readmission risk score and the determined AUC score to a clinician or other user via a user interface. The display may comprise information about the patient, the parameters, the input data for the patient, and/or the patient's readmission risk. Other information is possible. Alternatively, the report may be communicated by wired and/or wireless communication to another device. For example, the system may communicate the report to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands.

According to an embodiment, the display further includes information about an effect of one or more of the individual readmission prediction features in the complete set of readmission prediction features on the generated readmission risk score. For example, the display may comprise a SHAP value for one or more individual readmission prediction features.

According to an embodiment, the display may comprise a list, such as a ranked list, of one or more individual readmission prediction features with a highest effect on a higher readmission risk score for the patient. For example, the display may comprise the top three individual readmission prediction features with the highest impact on the increased probability of readmission. According to an embodiment, the display may comprise a list, such as a ranked list, of one or more individual readmission prediction features with a highest effect on a lowered readmission risk score for the patient. For example, the display may comprise the top three individual readmission prediction features with the highest impact on the decreased probability of readmission. Although three individual readmission prediction features are described in these examples, the actual number can be any number from zero to one to multiple features. According to an embodiment, determination of which individual readmission prediction feature(s) to display is based on comparing the effect of the prediction feature on a predetermined threshold, and displaying those features that meet and/or exceed that threshold. For example, a clinician may want to be notified of any prediction features that surpass a certain threshold, and thus this may be a setting or other variable parameter of the system.

At optional step 190 of the method, the clinician or other decisionmaker utilizes the displayed generated readmission risk score, the determined AUC score, and/or information about one or more individual readmission prediction features in patient care decision-making. For example, if the patient comprises a high readmission risk based on the displayed information, the clinician or other decisionmaker can consider, order, or otherwise implement an intervention for the patient to address the high readmission risk. For example, the decisionmaker can consider or implement decisions such as the patient discharge disposition, including whether the patient is discharged to home, or a care facility, or to another facility. The decisionmaker can consider or implement decisions such as follow-up frequency and/or type for the patient, home visits, social care visits, monitoring at home, and/or a lot of other decisions. These decisions, and their implementation, can be an attempt by the clinician to affect—and preferrable to lower—the readmission risk of the patient. The possible or potential effect of the decision on the readmission risk of the patient may be seen immediately or some time after the decision and/or implementation of the decision.

Referring to FIG. 2 is a schematic representation of a readmission risk analysis system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, the system may comprise, among other instructions or data, an electronic medical record system 270, training dataset 280, data processing instructions 262, training instructions 263, trained readmission risk model 264, and/or reporting instructions 265.

According to an embodiment, the electronic medical record system 270 is an electronic medical records database from which the information about the patient, including the plurality of readmission prediction features, may be obtained or received. The electronic medical records database may be a local or remote database and is in communication the patient risk score analysis system 200. According to an embodiment, the patient readmission risk analysis system comprises an electronic medical record database or system 270 which is optionally in direct and/or indirect communication with system 200.

According to an embodiment, the training data set 280 is a dataset that may be stored in a local or remote database and is in communication the patient readmission risk analysis system 200. According to an embodiment, the patient readmission risk analysis system comprises a training data set 280. The training data can comprise medical information about a patient, including but not limited to demographics, physiological measurements such as vital data, physical observations, and/or diagnosis, among many other types of medical information.

According to an embodiment, embodiment, data processing instructions 262 direct the system to retrieve and process input data which is used to train the readmission risk model 264. The data processing instructions 262 direct the system to for example, receive or retrieve input data or medical data to be used by the system as needed, such as from electronic medical record system 270 among many other possible sources. As described above, the input data can comprise a wide variety of input types from a wide variety of sources.

According to an embodiment, the data processing instructions 262 also direct the system to process the input data to generate a plurality of readmission prediction features related to medical information for a plurality of patients, which are used to train the classifier. This can be accomplished by a variety of embodiments for feature identification, extraction, and/or processing. The outcome of the feature processing is a set of readmission prediction features related to readmission risk analysis for a patient, which thus comprises a training data set that can be utilized to train the risk model 264.

According to an embodiment, training instructions 263 direct the system to utilize the processed data to train the readmission risk model 264. The readmission risk model can be any machine learning algorithm, classifier, or model sufficient to utilize the type of input data provided, and to generate a risk analysis. Thus, the system comprises a trained readmission risk model 264 configured to generate a readmission risk prediction or score for a patient, as described or otherwise envisioned herein.

According to an embodiment reporting instructions 265 direct the readmission risk analysis system to generate and provide a report to a user via a user interface comprising a generated readmission risk score and/or determined AUC score. According to an embodiment, the display may comprise information about the patient, the parameters, the input data for the patient, and/or the patient's readmission risk. Other information is possible. Alternatively, the report may be communicated by wired and/or wireless communication to another device. For example, the system may communicate the report to a mobile phone, computer, laptop, wearable device, and/or any other device configured to allow display and/or other communication of the report.

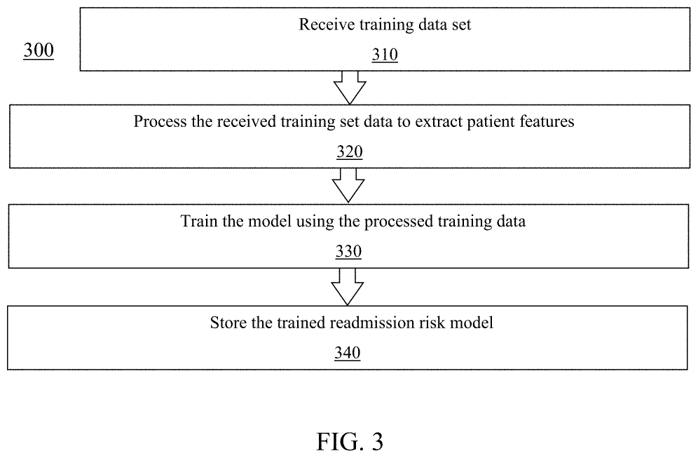

Referring to FIG. 3, in one embodiment, is a flowchart of a method 300 for training the readmission risk model of the patient readmission risk analysis system. At step 310 of the method, the system receives a

Referring to FIG. 3, in one embodiment, is a flowchart of a method 300 for training the risk model of the risk analysis system. At step 310 of the method, the system receives a training data set comprising training data about a plurality of patients. The training data can comprise medical information about each of the patients, including but not limited to demographics, physiological measurements such as vital data, physical observations, and/or diagnosis, among many other types of medical information. As an example, the medical information can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. Many other types of medical information are possible. According to an embodiment, the training data may also comprise an indication or information about one or more outcomes of each patient. According to an embodiment, the training data may comprise retrospective admission/discharge/transfer (ADT) data. The training data may be stored in and/or received from one or more databases. The database may be a local and/or remote database. For example, the patient readmission risk analysis system may comprise a database of training data.

According to an embodiment, the readmission risk analysis system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 320 of the method, the system processes the received information to extract readmission prediction features about one or more of the plurality of patients. The readmission prediction features may be any features which will be utilized to train the readmission risk model 264, such as any readmission prediction features that can or will be utilized by the trained algorithm for readmission risk analysis for a future patient. Feature extraction can be accomplished by a variety of embodiments for feature identification, extraction, and/or processing, including any method for extracting features from a dataset. The outcome of a feature processing step or module of the readmission risk analysis system is a set of readmission prediction features about a plurality of patients, which thus comprises a training data set that can be utilized to train the classifier.

According to just one embodiment, the readmission prediction features comprise one or more of the patient's: (i) length of stay (LOS); (ii) number of emergency department (ED) visits in a predetermined time period; (iii) acute admission status; (iv) age; (v) gender; (vi) number of elective admissions in a predetermined prior period; (vii) number of non-elective admissions in a predetermined prior period; and/or (viii) diagnosis (such as one or more ICD codes associated with the current patient for the current encounter; among many other possible readmission prediction features.

At step 330 of the method, the system trains the machine learning algorithm, which will be the algorithm utilized in analyzing patient information as described or otherwise envisioned. The machine learning algorithm is trained using the extracted features according to known methods for training a machine learning algorithm. According to an embodiment, the algorithm is trained, using the processed training dataset, to generate a readmission risk score and to determine an AUC score. According to an embodiment, the algorithm is trained, using the processed training dataset, to determine the effect of one or more of the individual readmission prediction features on the generated readmission risk score, such as through a SHAP value.

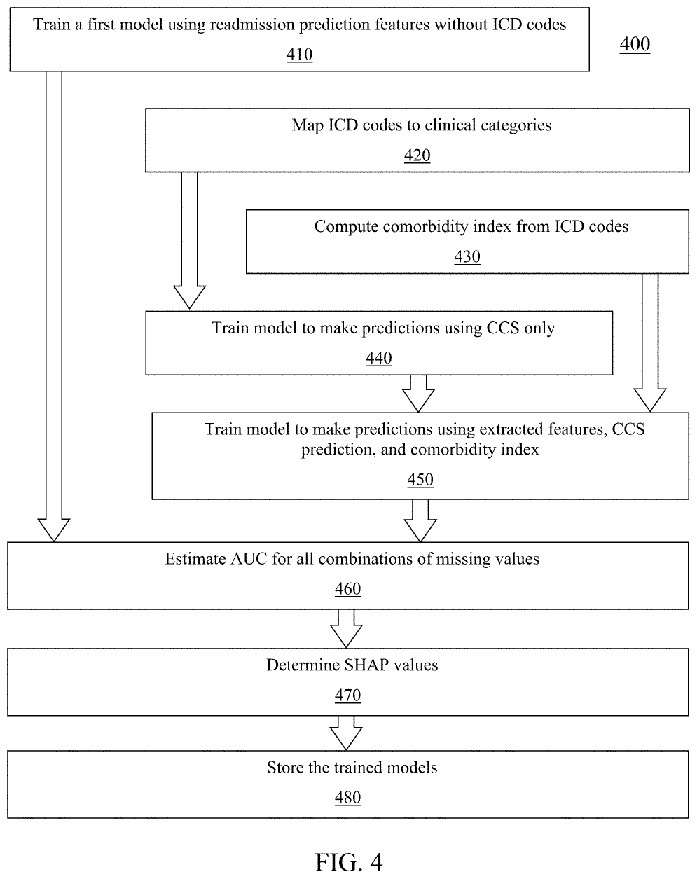

Referring to FIG. 4, in one embodiment, is a method 400 for training the machine learning algorithm, such as the training described in regard to step 330 of method 300 in FIG. 4. At step 410 of the method, the system trains the model or a model to predict a patient readmission risk using a plurality of readmission prediction features from the retrospective training data, where the plurality of readmission prediction features does not comprise ICD codes. Accordingly, the first trained model—the non-ICD code model—is trained to determine or estimate the readmission risk for a patient when there are no ICD codes available for the patient.

At step 420 of the method, the system maps ICD codes in the patient data set to clinical categories (CCS), thus reducing the ICD codes from many thousands to potentially just hundreds. According to an embodiment, the system utilizes a private algorithm or a public tool to map the ICD codes to CCS. For example, the system may utilize the Clinical Classifications Software Refined (CCSR) tool from the Healthcare Cost and Utilization Project (HCUP) to map the ICD codes to CCS, although many other tools and methods are possible.

According to an embodiment, the system utilizes diagnosis (ICD codes), as prediction features in a way that keeps the model interpretable and user friendly, since there are about 60,000 different ICD codes. According to an embodiment, the system maps ICD codes to approximately 400 clinical categories, wherein each code might belong to x different categories, for example. The system can create an encoder data frame where each column is a clinical category and each row represents one encounter.

At step 430 of the method, the system calculates a comorbidity score or index for each patient in the retrospective training data, using the readmission prediction features extracted from that retrospective training data, using the ICD codes in the patient data set. The system may utilize any method for calculating a comorbidity score or index for the patient. According to an embodiment, the system may utilize a comorbidity system such as the Charleston comorbidity system, the Elixhauser (van Walraven) comorbidity system, or any other method or system. The system maps ICD codes to comorbidities, then maps each comorbidity to a score and sums them per encounter.

At step 440 of the method, the system trains a second model—the ICD code model—to predict a patient readmission risk using only the clinical categories (CCS). Accordingly, the model is trained to determine or estimate the readmission risk for a patient when there are ICD codes, mapped to CCS, available for the patient. This predicted patient readmission risk is utilized in the next step of the method.

At step 450 of the method, the system trains the second model—the ICD code model— to predict a patient readmission risk using a plurality of readmission prediction features from the retrospective training data, the computed comorbidity index, and the CCS prediction from step 440 of the method. Accordingly, the second trained model—the ICD code model—is trained to determine or estimate the readmission risk for a patient when there are ICD codes available for the patient.

According to just one embodiment, the readmission prediction features comprise one or more of the patient's: (i) length of stay (LOS); (ii) number of emergency department (ED) visits in a predetermined time period; (iii) acute admission status; (iv) age; (v) gender; (vi) number of elective admissions in a predetermined prior period; (vii) number of non-elective admissions in a predetermined prior period; and/or (viii) diagnosis (such as one or more ICD codes associated with the current patient for the current encounter; among many other possible readmission prediction features.

Accordingly, the system will comprise two trained models, a non-ICD code model and an ICD code model. For future patients for which the models will be utilized to estimate readmission risk, the system can utilize either model. For example, if ICD code(s) are present for the patient, the system can compute a CCS score and a comorbidity score, and can predict the risk of readmission using the ICD code model. For example, if ICD codes are missing for the patient, the system can predict the risk of readmission using the non-ICD code model.

At step 460 of the method, the system estimates an area under the curve (AUC) score for all combinations of missing values for the plurality of readmission prediction features. According to an embodiment, the system utilizes a pre-populated lookup table of the readmission risk analysis system to retrieve information and generate an AUC score. The pre-populated lookup table comprises an AUC score for a plurality of sets of readmission prediction features, where each set of the plurality of sets comprises one or more missing readmission prediction features. According to an embodiment, this step comprises performance estimation in the presence of missing data.

According to an embodiment, the trained models support both missing features and features with missing values, meaning that for any set of inputs a prediction will be made. The accuracy of each prediction (as measured by AUC) depends on availability of input readmission prediction features for a patient. Thus, the system may comprise a model that is able to determine or estimate accuracy depending upon the availability of input readmission prediction features for a patient.

According to an embodiment, the average AUC of each combination of available features (512 permutations) was estimated retrospectively using test data and stored as a Jason file, although other methods are possible. In this method, the readmission risk analysis comprises nine (9) readmission risk prediction values that are utilized for a readmission risk analysis for a patient. Accordingly, the system creates a data frame of 512 rows and 9 columns, where each row is a unique combination of True/False. For each perm in permutations: (a) set temp data=data[perm]=NA; (b) predict readmission risk using temp data; and (c) compute AUC. The system can create a dictionary with permutations as keys and AUC as values. The system can thus comprise a lookup table, dictionary, or index for missing data and corresponding AUC values.

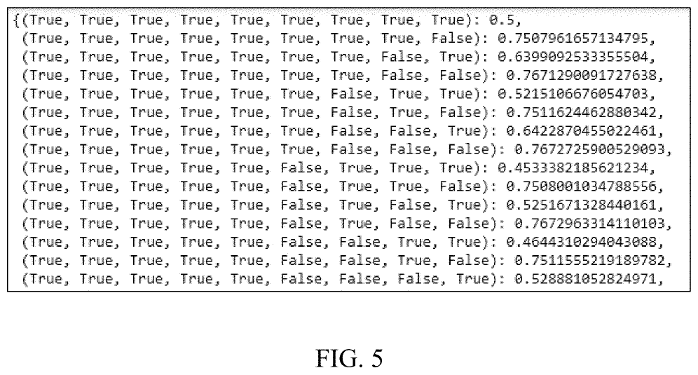

Referring to FIG. 5, for example, is the beginning of the data frame of 512 rows with 9 columns and the generated AUC score. In row #1 all data is available, and the corresponding AUC value is 0.5. In row #2, the ninth readmission prediction value is missing or missing a usable value, and thus is marked “false” and the corresponding AUC value is approximately 0.7508, and so on.

At step 470 of the method, the system computes SHAP (SHapley Additive exPlanation) values for each of the individual readmission prediction features. The SHAP value may be determined using any method for determining SHAP values, or otherwise generating, calculating, estimating, or otherwise determining the effect.

Following these steps of the method, the patient readmission risk analysis system comprises trained algorithms or models or classifiers that can be utilized to generate a readmission risk analysis as described or otherwise envisioned. The trained classifiers can be static such that they trained once and are utilized for classifying. According to another embodiment, the trained classifiers can be more dynamic such that they are updated or re-trained using subsequently available training data. The updating or re-training can be constant or can be periodic.

At step 480 of the method, the trained algorithms can be stored locally or remotely for subsequent analysis of patient features.

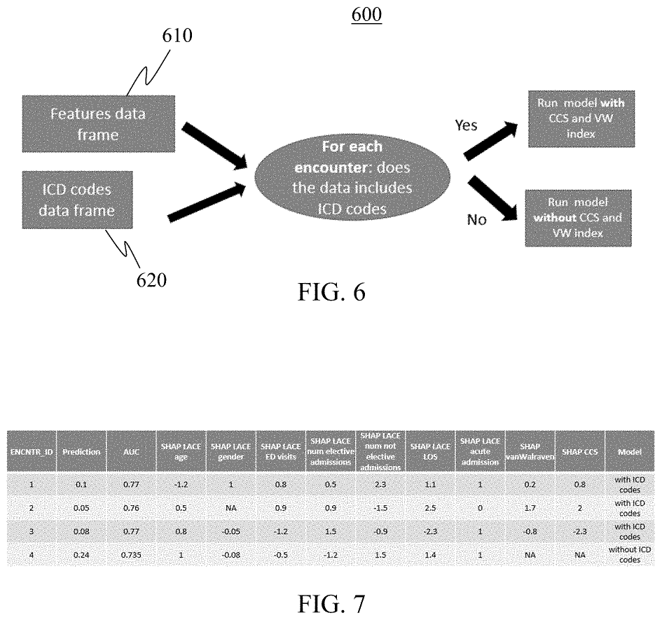

Referring to FIG. 6, in one embodiment, is a flowchart of a method 600 for determining a readmission risk for a patient using the readmission risk analysis system. The input for the risk determination comprises either a data frame with ICD codes 610, or a data frame without ICD codes 620. The system determines whether the input comprises ICD codes, in order to determine which trained model is used to perform the readmission risk analysis. If the input comprises ICD codes (“yes”), then the ICD code model is utilized, and the CSS and comorbidity index (“VW) is utilized as well. If the input does not comprise ICD codes (“no”), then the non-ICD code model is utilized, and the CSS and comorbidity index (“VW) are similarly not utilized.

Referring to FIG. 7, in one embodiment, is an output of patient readmission risk analysis for four patients. ICD codes were available for patients (“ENCNTR_ID”) 1 through 3, but were not available for patient 4. Thus, the ICD code model with the CSS and comorbidity index was utilized for patients 1 through 3, and the non-ICD code model without the CSS and comorbidity index was utilized for patient 4. The output comprises a readmission risk prediction (“Prediction”) and AUC score (“AUC”) for each patient regardless of model used. The output also comprises a SHAP value for each readmission prediction feature utilized in the analysis.

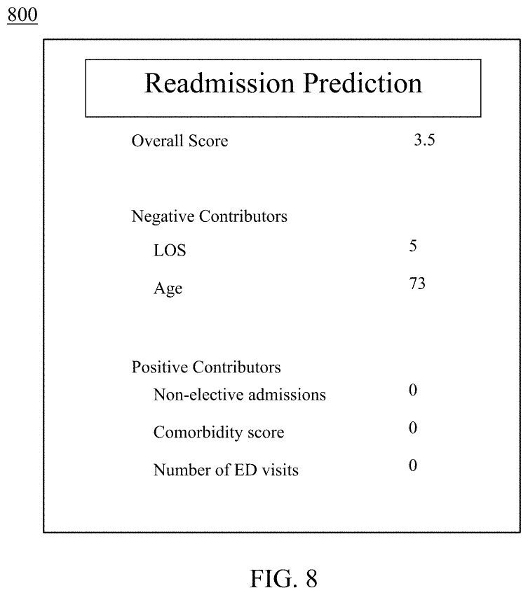

Referring to FIG. 8, in one embodiment, is an output 800 of a patient readmission risk analysis for a patient, comprising a Readmission Prediction. This may be an example of a visualization provided to a user, although many other visualizations and variations are possible. The Readmission Prediction comprises an Overall Score of 3.5, which may be on a scale of 0-5, 0-10, 0-100, or any other scale. The Overall Score comprises Negative Contributors, which increase the likelihood of readmission risk, and Positive Contributors, which decrease the likelihood of readmission risk. In this example, the Negative Contributors comprise the length of state (LOS) of 5 days, and the age of the patient at 73 years. In this example, the Positive Contributors comprise no non-elective admissions, no ED visits, and a comorbidity score of 0.

According to an embodiment, the patient readmission risk analysis system is configured to process many thousands or millions of datapoints in the input data used to train the classifier, as well as to process and analyze the received plurality of patient features. For example, generating a functional and skilled trained classifier using an automated process such as feature identification and extraction and subsequent training requires processing of millions of datapoints from input data and the generated features. This can require millions or billions of calculations to generate a novel trained classifier from those millions of datapoints and millions or billions of calculations. As a result, each trained classifier is novel and distinct based on the input data and parameters of the machine learning algorithm, and thus improves the functioning of the patient readmission risk analysis system. Thus, generating a functional and skilled trained classifier comprises a process with a volume of calculation and analysis that a human brain cannot accomplish in a lifetime, or multiple lifetimes.

In addition, the patient readmission risk analysis system can be configured to continually receive patient features, perform the analysis, and provide periodic or continual updates via the report provided to a user for the patient. This requires the analysis of thousands or millions of datapoints on a continual basis to optimize the reporting, requiring a volume of calculation and analysis that a human brain cannot accomplish in a lifetime.

By providing an improved patient readmission risk analysis, this novel patient readmission risk analysis system has an enormous positive effect on patient readmission risk analysis compared to prior art systems. As just one example in a clinical setting, by providing a system that can improve patient readmission risk analysis with confidence intervals, the system can facilitate treatment decisions and improve survival outcomes, thereby leading to saved lives.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for generating and presenting a patient readmission risk using a readmission risk analysis system, comprising:
receiving, at the readmission risk analysis system, information about the patient, wherein the information comprises a plurality of readmission prediction features;
extracting, by a processor of the readmission risk analysis system, the plurality of readmission prediction features from the received information;
analyzing, by the processor, the extracted plurality of readmission prediction features to determine whether each of a predetermined list of readmission prediction features are present;
replacing one or more identified missing readmission prediction features with a null value to generate a complete set of readmission prediction features for the patient;
analyzing, using a trained machine learning algorithm of the risk score analysis system, the complete set of readmission prediction features for the patient to generate a readmission risk score;
determining, using a populated lookup table of the readmission risk analysis system, an AUC score, wherein the populated lookup table comprises an AUC score for a complete set of readmission prediction features when the complete set of readmission prediction features comprises the one or more identified missing readmission prediction features; and
displaying, via a user interface of the readmission risk analysis system, the generated readmission risk score and the determined AUC score.

2. The method of claim 1, wherein the display further comprises an effect of one or more of the individual readmission prediction features in the complete set of readmission prediction features on the generated readmission risk score.

3. The method of claim 2, wherein the display of the effect of one or more of the individual readmission prediction features on the generated readmission risk score comprises a ranked list of: (1) one or more individual readmission prediction features on with a highest effect increasing the readmission risk score for the patient; or (2) one or more readmission prediction features on with a highest effect decreasing the readmission risk score for the patient.

4. The method of claim 3, wherein the highest effect on a higher readmission risk score for the patient or the highest effect on a lower readmission risk score for the patient is determined by comparing the effect to a predetermined threshold.

5. The method of claim 1, wherein the plurality of readmission prediction features comprises a diagnosis for the patient.

6. The method of claim 1, wherein the readmission risk score comprises a SHAP value for one or more of the individual readmission prediction features in the complete set of readmission prediction features.

7. The method of claim 1, wherein displaying further comprises displaying a SHAP value for one or more of the individual readmission prediction features in the complete set of readmission prediction features.

8. The method of claim 1, further comprising training a model of the readmission risk analysis system using a training dataset comprising data about a plurality of patients, comprising:
training a first model of the readmission risk analysis system to generate a first model readmission risk score without a diagnosis information for a patient;
mapping each of a plurality of ICD codes to one or more of a plurality of clinical categories;
computing, from the training dataset, a comorbidity index for each patient in the plurality of patients;
training an intermediate model of the readmission risk analysis system to generate, for each patient, an intermediate clinical category-based readmission risk score using the plurality of clinical categories;
training a second model of the readmission risk analysis system to generate a second model readmission risk score for each of the plurality of patients using, for each patient: (i) a plurality of prediction features extracted from the training data for the respective patient; (ii) the generated intermediate clinical category-based readmission risk score for the respective patient; and (iii) the computed comorbidity index for the respective patient.

9. The method of claim 8, further comprising:
estimating an AUC score for all possible combinations of missing values for the plurality of prediction features; and
determining a SHAP value for each of the plurality of prediction features.

10. A readmission risk analysis system configured to generate and present a patient readmission risk for a patient, the system comprising:
a trained readmission risk model configured to generate a readmission risk score from a plurality of extracted readmission prediction features about a patient;
a processor configured to: (i) receive information about the patient, wherein the information comprises a plurality of readmission prediction features; (ii) extract the plurality of readmission prediction features from the received information; (iii) analyze the extracted plurality of readmission prediction features to determine whether each of a predetermined list of readmission prediction features are present; (iv) replace one or more identified missing readmission prediction features with a null value to generate a complete set of readmission prediction features for the patient; (v) analyze, using the trained readmission risk model, the complete set of readmission prediction features for the patient to generate a readmission risk score; and (vi) determining an AUC score; and
a user interface configured to present to a user the generated readmission risk score and AUC score for the patient.

11. The system of claim 10, wherein the user interface is further configured to display an effect of one or more of the individual readmission prediction features in the complete set of readmission prediction features on the generated readmission risk score.

12. The system of claim 11, wherein the display of the effect of one or more of the individual readmission prediction features on the generated readmission risk score comprises a ranked list of: (1) one or more individual readmission prediction features on with a highest effect increasing the readmission risk score for the patient; or (2) one or more readmission prediction features on with a highest effect decreasing the readmission risk score for the patient.

13. The system of claim 10, wherein the plurality of readmission prediction features comprises a diagnosis for the patient.

14. The system of claim 10, wherein the readmission risk score comprises a SHAP value for one or more of the individual readmission prediction features.

15. The system of claim 10, wherein the user interface is further configured to display a SHAP value for one or more of the individual readmission prediction features.
